(19) United States
(12) Patent Application Publication
(10) Pub. No.: US 2022/0257641 A1
(43) Pub. Date: Aug. 18, 2022

(54) A COMPOSITION COMPRISING SIMETHICONE AND SUCROSE ESTERS AND USE THEREOF AS AN ANTIFOAM AGENT

(71) Applicant: S.I.I.T. S.R.L. SERVIZIO INTERNAZIONALE IMBALLAGGI TERMOSALDANTI., Trezzano sui Naviglio (MI) (IT)

(72) Inventors: Andrea Domizio COSTA, Trezzano sui Naviglio (MI) (IT); Elena MADARO, Trezzano sui Naviglio (MI) (IT); Mirko DOMINONI, Trezzano sui Naviglio (MI) (IT)

(21) Appl. No.: 17/628,184

(22) PCT Filed: Jul. 24, 2020
(86) PCT No.: PCT/IB2020/057030

§ 371 Date: Jan. 18, 2022

(30) Foreign Application Priority Data
Jul. 24, 2019 (IT) 102019000012804

(51) Int. Cl. A61K 31/80; A61K 31/7024; A61K 9/00; A61P 1/14; A23L 33/00; A23L 33/19

(52) U.S. Cl. CPC A61K 31/80; A61K 31/7024; A61K 9/0095; A23V 2002/00; A23L 33/40; A23L 33/19; A61P 1/14

(57) ABSTRACT

The present invention relates to a composition, preferably for oral use, comprising a simethicone and sucrose esters (sucrester) and, optionally, at least one food or pharmaceutical grade additive and/or excipient. Furthermore, the present invention relates to said composition for use in a method for the treatment of disorders of the gastrointestinal tract, in particular of intestinal colics, especially in paediatric subjects and in neonates, abdominal cramps, flatulence, tympanites, aerophagia, swelling of the abdomen, dyspeptic disorders (or dyspepsia) and disorders that require a reduction in gastric emptying time. Lastly, the present invention relates to the non-therapeutic use of said composition as antifoam.

The present invention relates to a composition, preferably for oral use, comprising a mixture comprising or, alternatively, consisting of simethicone and sucrose esters (or sucrester) and, optionally, said composition comprises at least one food or pharmaceutical grade additive and/or excipient. Furthermore, the present invention relates to said composition for use in a method for preventive and/or curative treatment of disorders of the gastrointestinal tract, in particular of intestinal colics, especially in paediatric subjects and in neonates, abdominal cramps, flatulence, tympanites, aerophagia, swelling of the abdomen, dyspeptic disorders (or dyspepsia) and disorders that require a reduction in gastric emptying time. Lastly, the present invention relates to the non-therapeutic use of said composition as antifoam.

The formation of foam with dense bubbles or gas bubbles covered by mucus that develop in the gastrointestinal tract of a subject arise from various causes. For example, proteins (for example, milk proteins) ingested through feeding produce foam in the gastrointestinal tract; or, swallowing air by eating too quickly or ingesting foods that cause flatulence, increases air in the gastrointestinal tract that leads to formation of foam. In such circumstances, the air retained in the form of foam cannot be disposed of in a natural manner or absorbed by the body. Furthermore, said foam slows digestion and creates gastrointestinal problems such as, for example, intestinal or abdominal colics, abdominal cramps, especially in children and neonates (or infants), flatulence, swelling of the abdomen or of gastrointestinal tract, dyspeptic disorders (or dyspepsia), increased gastric emptying time.

The number of people affected by these disorders is high, particularly children and neonates with intestinal colics (neonatal colics). Thus, there is a high market demand for a solution to these disorders.

Colic or colic pain defines a specific form of perception of pain characterised by paroxysmal accesses interspaced with periods of remission. As a characteristic symptom of hollow organ diseases, pain is present in the phases of bowel contraction, while remission is present in the phases of relaxation. Abdominal (or intestinal) colic is used to indicate the cramp-like pain located in the lower abdominal area where the intestine is located. In general, abdominal colics are more or less serious depending on the factor that triggers it. There is a tendency to hardening on the abdominal area, discomfort, pain and swelling in most cases with discomfort even to the touch. Abdominal (or intestinal) colics are common in neonates due to their still incomplete digestive function.

Dyspepsia (or dyspepsia disorders) indicates a pathological condition characterised by the predominant presence of persistent or recurrent pain and/or discomfort located in the epigastrium, more or less associated with sense of fullness and anorexia. Since clinical parameters that allow to distinguish healthy subjects from those suffering from dyspeptic diseases have not been defined, it is not possible to classify different symptomatic cases in precise clinical pictures. Pictures with dyspepsia are normally classified based on symptoms, such as bloating, feeling of stomach fullness, eructation, nausea or heartburn.

The use of compositions comprising a simethicone for the treatment of intestinal colics, in particular in paediatric subjects or neonates is known in the prior art, such as for example the patent document IT NA20100053, in which a composition comprising a simethicone and a Saccharomyces acts by restoring the gut microbiota balance in order to resolve gastrointestinal disorders, also including colics. As known, simethicone acts by means of purely mechanical action on the formation of gas in the gastrointestinal tract, dissolving the gas produced. The use of simethicone alone did not give in the known products the desired efficacy in the dissolution of the gas that is formed in the intestine, in particular in paediatric subjects or neonates and therefore in the resolution of gastrointestinal disorders associated with the formation of gas in the gastrointestinal tract (or intestinal tract).

The present invention addresses and solves the problem of providing an effective composition in the treatment of diseases, disorders or symptoms caused by or related with the increase in the pressure of the gastrointestinal tract due to excess gases, in particular in paediatric subjects (preferably subjects from 1 month to 18 months, more preferably from 1 month to 6 or 12 months) and in neonates (subjects from birth to 4 weeks). Said excess gas in the gastrointestinal tract which creates the aforementioned disorders is at least partly due to the ingestion and digestion of proteins, in particular milk proteins. As a matter of fact, it is assumed that in neonate or paediatric subjects fed fully or partly based on milk (breast milk or artificial milk) the production of gas (CO₂) is mainly related to the digestion of milk proteins, besides the ingestion of air during suction.

Following an intense research and development phase, with the present invention the Applicant provides a composition with antifoam effect which is capable of breaking the foam generated in the gastro-intestinal tract, so as to release the gas “trapped” in the foam. Thus, the gas, no longer in the form of foam, can escape from the gastrointestinal tract in a natural way or be absorbed by the body.

By virtue of its effectiveness as an antifoam agent, the composition of the invention provides an effective solution to the diseases, disorders or symptoms caused by or related with the increase in the pressure of the gastrointestinal tract due to excess gas.

The composition of the invention is effective in the treatment of disorders such as colics and abdominal (or intestinal) cramps due to foam generated by proteins ingested by a subject, in particular milk proteins, and therefore to treat colics in infants (children and neonates).

The present invention combines in a single product two compounds of the partially superimposable mechanism of action (i.e. mechanical breakage of air bubbles present on the surface of gastrointestinal fluids), but with a differentiated activity target: a simethicone which acts generically on all gastrointestinal foams, and a sucrester which acts specifically on the protein foams present in the gastrointestinal tract (foams due to ingestion and digestion of proteins, preferably milk proteins).

Furthermore, when the composition of the invention is administered to a paediatric subject or neonate in a mixture with milk (e.g. powdered milk dispersed in water), the sucrester present in the composition reduces the formation of milk foam which is created upon mixing powdered milk and water. It has been hypothesised that the effectiveness of the sucresters used in the present invention in reducing the foams of a protein nature is due to their ability to significantly reduce the surface tension of the bubbles of said foams of a protein nature at the water-air interface.

Furthermore, the composition of the invention does not reveal adverse effects and it can be administered to all subjects, in particular even to paediatric subjects and neonates. Sucresters are food additives identified with n° E473 which are permitted as additives in infant formulations (EU Regulation 1333/2008). The amount of simethicone comprised in a therapeutically effective amount of the composition of the invention (effective daily intake) is less than the amounts of simethicone comprised in the compositions currently available for use in the treatment of similar disorders and diseases.

Lastly, the composition of the invention is easy to prepare and economically advantageous.

In addition, the composition of the invention is capable of treating gastrointestinal disorders through the protection of the gastrointestinal wall.

### FIGURES

FIG. 1: illustration of the method used to determine the antifoam effect using a Minijet™ device (trademark registered by Teclis).

FIGS. 2, 3 and 4: comparison of the foam volumes with respect to the time in the experiments conducted using the method illustrated in FIG. 1.

FIGS. 5A, 5B, 5C, 5D, 5E, 5F: visual comparison of the foams formed in a mixture of water and powdered milk in the absence of simethicone at zero time (5A, t₀), and following the addition of simethicone alone in aqueous solution to the mixture: at zero time (t₀, 5B), after 2 minutes (t1, 5C), after 4 minutes (t2, 5D), after 6 minutes (t3, 5E), and after 8 minutes (t4, 5F).

FIGS. 6A, 6B, 6C, 6D, 6E, 6F: visual comparison of the foams formed in a mixture of water and powdered milk in the absence of simethicone at zero time (6A, t₀), and following the addition of simethicone alone in oily solution, respectively at zero time (t0, 6B), after 2 minutes (t1, 6C), after 4 minutes (t2, 6D), after 6 minutes (t3, 6E), and after 8 minutes (t4, 6F).

FIGS. 7A, 7B, 7C, 7D, 7E, 7F: visual comparison of the foams formed in a mixture of water and powdered milk in the absence of simethicone at zero time (7A, t₀), and following the addition of a composition according to the invention comprising a simethicone and a sucrester in aqueous solution, respectively at zero time (t0, 7B), after 2 minutes (t1, 7C), after 4 minutes (t2, 7D), after 6 minutes (t3, 7E), and after 8 minutes (t4, 7F).

FIG. 8A: photo of a mixture of water and powdered milk in the presence of a composition comprising simethicone alone in aqueous solution: positioning the composition comprising simethicone in the interface between liquid and foam.

FIG. 8B: photo of a mixture of water and powdered milk in the presence of a composition comprising simethicone alone in oily solution: positioning the composition comprising simethicone over the foam.

FIG. 9A: photo of a mixture of water and powdered milk in the presence of the composition according to the invention.

FIG. 9B: photo of a mixture of water and powdered milk in the absence of the composition according to the invention.

### DETAILED DESCRIPTION OF THE INVENTION

Forming an object of the present invention is a composition (in short, composition of the invention) comprising, or alternatively, consisting of a simethicone and sucrose esters (or a sucrester) and, optionally, said composition comprises at least one food or pharmaceutical grade additive and/or excipient.

The term “simethicone” (example CAS 8050-81-5), alternatively called activated dimethicone, refers to a polydimethylsiloxane (or dimethicones, (C₂H₆OSi)ₙ, example CAS 63148-62-9) activated with silica dioxide (synthetic polymer). Simethicones have structural formula (C₂H₆OSi)ₙ(SiO₂)ₘ with n and m variable.

In a preferred embodiment, the simethicone comprised in the composition of the invention, together with the esters of fatty acids, is in the form of simethicone emulsion from 10% to 35% weight/weight (in short, w/w), preferably from 15% or 20% to 35% w/w, more preferably from 25% to 35% w/w.

The expression “simethicone emulsion from x % to y % w/w” is used to indicate a simethicone dispersed in water (simethicone-water emulsion) with an active content (i.e. simethicone) comprised in the range from x % to y % by weight with respect to the total weight of the simethicone-water emulsion.

For example, the expression “simethicone emulsion at 25%-35%” is used to indicate a simethicone dispersed in water (simethicone-water emulsion) with an active content (i.e. simethicone) comprised in the range from 25% to 35% (e.g. 27%, 29%, 30%, 31% or 33%) by weight with respect to the total weight of the simethicone-water emulsion, preferably from 27.5% to 32.5%, more preferably about 30%, and a density at 25° C. (P=1 atm) comprised from 0.80 g/cm3 to 1.20 g/cm3, preferably of about 1 g/cm³ (0.98-1.010 g/cm³) (density measured using standard equipment and methods, e.g. DIN 51757 method).

Examples of simethicone (simethicone emulsion from 25% to 35% w/w) usable in the mixture and/or in the composition of the present invention, together with a sucrester, are:

i) Dow Corning® Q7-2587 30% (Simethicone emulsion USP): (contains 30% by weight of USP (United States Pharmacopeia) simethicone; stearate as emulsifier, sorbic acid, benzoic acid, thickeners and water):
pH=2.6;
Defoaming performance at 50 ppm=less than 15 seconds;
Polydimethylsiloxane content=30.4%;
Heavy metal content=less than 5 ppm.

ii) SILFAR®SE4 (silicone antifoam emulsion):
Density at 25° C. (DIN 51757 method)=1 g/cm³;
Solid constituent (microwave method)=31-34%;
“Simethicone” active ingredient, USP/EP=30%;
pH (Indicator strips method)=3-5.

The percentages reported in the products (i) and (ii) are percentages by weight with respect to the total weight of the products.

The simethicone present in the composition of the invention is not absorbed by the gastrointestinal tract and it does not interfere with the absorption of nutrients. It does not alter the volume and the acidity of gastric secretions and chronic toxicity studies in rats have shown that the absorption of essential metabolites does not decrease. Thus, the composition of the invention does not reveal adverse effects and it can be administered to all subjects, in particular even to paediatric subjects and neonates.

The term “sucrose esters”, alternatively referred to as sucrester or sucresters or sucrose esters of fatty acids or ester of a carbohydrate with one or more fatty acids, is used to indicate sucrose esters with fatty acids having a number of carbon atoms comprised in the range from C6 to C12 (middle-chain fatty acids), straight or branched, saturated or unsaturated, or, alternatively, having a number of carbon atoms comprised in the range from C13 to C21 (long-chain fatty acids), preferably C16-C18, straight or branched, saturated or unsaturated.

The sucresters are obtained from the esterification of the fatty acids or from the trans-esterification of the methyl esters of the fatty acids with carbohydrates (also called saccharides). Sucrose is generally the carbohydrate used. This is why sucresters are also referred to as sucrose esters of fatty acids. The chemical/physical properties of these compounds depend on the number and the type of esterified fatty acids.

Preferably, the sucrose esters or sucrester/s present in the mixture and/or in the composition of the present invention in the embodiments thereof are a sucrester/s E473. The initials E473 indicates that the sucresters are food grade additives allowed by the European law and regulated by the Ministerial Decree (D.M.1996).

In an embodiment, said sucrose esters or sucrester, preferably sucrester E473, present in the mixture or composition of the invention together with a simethicone, comprise sucrose esters at least at a % by weight comprised in the range from 70% to 98%, preferably from 85% to 95%, more preferably about 90%, obtained through the esterification of sucrose with fatty acids of plant origin, saturated or unsaturated, having a number of carbons comprised in the range from C6 to C22, preferably from C12 to C22, more preferably C16 and C18 (for example stearic acid and palmitic acid). The remaining percentage by weight mainly comprises free fatty acids and/or unesterified sucrose.

Preferably, said sucrose esters or sucrester, preferably sucrester E473, present in the mixture or composition of the invention together with a simethicone, comprise fatty acid sucrose monoesters having a number of carbons C16 e/o C18 at a percentage by weight from 50% to 90%, preferably from 60% to 80%.

For example, sucrose esters or sucrester usable in the mixture and/or composition of the present invention, together with a simethicone, may have the following composition by weight: a total ester content of at least 90%, of which a monoester content of 70% obtained through the esterification of sucrose with fatty acids of plant origin (mainly stearic acid and palmitic acid); a free fatty acid content not exceeding 3% (as oleic acid); free sucrose content not exceeding 2%; maximum p-methoxyphenol 100 microg/kg; maximum acetaldehyde 50 ppm; humidity not exceeding 4%; acid value not exceeding 5 expressed as mg of KOH/g (example of commercial product: sucrose esters SP70 ‘E473’, marketed by Brenntag). Sucrose esters of fatty acids are comprised in annex II to EC Regulation 1129/2011 and in EC Regulation 231/2012 under item E473.

In an embodiment, the mixture contained in the composition of the invention comprises simethicone in aqueous emulsion from 25% to 35% w/w, preferably at about 30% w/w, and sucrose esters, preferably sucrose esters E473 predominantly of palmitic acid and stearic acid.

Preferably, in the composition of the invention simethicone and sucrose esters are in a [simethicone as such: sucrose esters] by weight ratio comprised in the range from 15:1 to 1:1 (for example 12:1, 10:1, 8:1, 7:1 or 6:1), preferably from 5:1 to 3:1, more preferably 4:1.

In the context of the present invention, when defining a by weight ratio or a % by weight of “simethicone as such”, it is used to indicate the weight of the simethicone present in a commercial simethicone product. For example, the commercial product “simethicone emulsion at 30% w/w” means that said commercial product contains 30% by weight of simethicone with respect to the total weight of the product which could contain water but also other additives.

In a preferred embodiment, the mixture of the invention comprises simethicone in aqueous emulsion from 25% to 35% w/w (or from 10% to 35% w/w), preferably at about 30% w/w, and sucrose esters, preferably sucrose esters of palmitic and stearic acid, in a [simethicone as such: sucrose esters] by weight ratio=from 15:1 to 1:1, preferably from 5:1 to 3:1, more preferably 4:1.

Preferably, the composition of the invention comprises simethicone and sucrose esters in percentages by weight with respect to the total weight of the composition comprised in the range (a) for simethicone (understood as simethicone as such) from 0.5% or 1% to 10% (for example 2%, 4%, 6% or 8%), preferably from 1% to 7%, more preferably from 3% to 5% (for example 4%), and (b) for sucrose esters of fatty acids from 0.05% or 0.1% to 1.5%, preferably from 0.5% to 1.5%, more preferably from 0.8% to 1.2% (for example 1%), and, optionally, for a stabiliser (c) selected from group A from 0.05% to 0.5%, preferably from 0.1% to 0.5%, more preferably about 0.5%. The remaining percentage of the composition preferably comprises water and additives and/or excipients. For example, simethicone as such from 1% to 7%, fatty acid esters from 0.1% to 1.5% and, optionally, a stabiliser selected from group A from 0.1% to 0.5%; preferably simethicone as such from 3% to 5%, fatty acid esters from 0.8% to 1.2% and, optionally, a stabilizer selected from the group A about 0.5%.

In a preferred embodiment, the composition of the invention comprises simethicone in aqueous emulsion from 25% to 35% w/w, preferably at about 30% w/w, and sucrose esters, preferably sucrose esters of palmitic and stearic acid, in percentages by weight with respect to the total weight of the composition comprised in the range (a) for the simethicone as such from 0.5% or 1% to 10% (for example 2%, 4%, 6% or 8%), preferably from 1% to 7%, more preferably from 3% to 5% (e.g. 4%), and (b) for sucrose fatty acid esters from 0.05 or 0.1% to 1.5%, preferably from 0.5% to 1.5%, more preferably from 0.8% to 1.2% (e.g. 1%), and, optionally, a stabiliser selected from group A from 0.05% to 0.5%, preferably from 0.1% to 0.5%, more preferably about 0.5%. The remaining percentage of the composition preferably comprises water and additives and/or excipients.

In an embodiment, besides a simethicone and sucrose esters, the composition of the invention comprises at least one further active component selected from the group comprising or, alternatively, consisting of: enzymes, direct or indirect antacid action substances, thickeners, dispersible solids (powders), prebiotic substances, probiotic substances belonging to the families of yeasts and bacteria, immunostimulant substances, antidiarrheal substances, nutritional substances, vitamins of group B, C, D, E, organic and/or inorganic salts of magnesium, selenium, zinc, melatonin, valerian, passion flower, lemon balm, hawthorn, chamomile, hop plant, antioxidants, anti-radical agents, substances with gastroprotective activity and mixtures thereof.

In an embodiment, besides a simethicone and sucrose esters, the mixture contained in the composition of the invention comprises (c) a high molecular weight (preferably neutral) polysaccharide with a barrier effect for the protection of the gastrointestinal wall and with the function of stabilising the composition of the invention, wherein said (c) polysaccharide (or stabiliser) is preferably selected from a group A comprising or, alternatively, consisting of: xanthan gum, gum karaya, gum arabic, tara gum and mixtures thereof. The term “high molecular weight polysaccharide” is used to indicate a polysaccharide with a molecular weight comprised from 500 kDa to 10000 kDa, preferably from 700 kDa to 5000 kDa, more preferably from 1000 kDa to 2500 kDa.

According to an example, the composition of the invention comprises simethicone in aqueous emulsion from 25% to 35% w/w (e.g. about 30% w/w), sucrose esters (mainly palmitic and stearic acid), a stabiliser selected from group A (preferably xanthan gum), water and excipients in the following percentages by weight with respect to the total weight of the composition: (a) simethicone as such from 1% to 10%, (b) sucrose esters from 0.05% to 1.5%, (c) stabiliser from 0.05% to 0.5%, (d) demineralised water from 75% to 95% and the remaining percentage to 100% excipients; preferably (a) from 1% to 7%, (b) from 0.5% to 1.5%, (c) from 0.1% to 0.5%, (d) about 85%, and the remaining percentage to 100% excipients; more preferably (a) from 3% to 5% (e.g. 4%), (b) from 0.8% to 1.2% (e.g. 1%), (c) about 0.5%, (d) about 85%, and the remaining percentage to 100% excipients.

The composition of the invention, comprising a simethicone and sucrose esters in the various embodiments of the invention, may be in solid form, such as tablet, chewable tablet, capsule, lozenge, granules, flakes or powder, in semi-solid form, such as soft-gel, or in liquid form, such as solution, suspension, dispersion, emulsion or syrup; preferably it is in liquid form, more preferably in liquid form of suspension in water or in oil (for example olive oil).

Advantageously, the composition of the invention is in liquid form in aqueous solvent (water), preferably in liquid form of suspension in aqueous solvent (water).

Alternatively, the composition of the invention may be in liquid form in a hydroalcoholic solvent.

Advantageously, the composition of the invention can be formulated for oral (or gastroenteric) use (or administration), preferably for oral use in liquid form, more preferably for oral use in liquid form suitable for administration as drops, sprays or syrup.

In a preferred embodiment, the composition of the invention, comprising a simethicone and sucrose esters in the various embodiments of the invention, is in liquid form of aqueous suspension (water) suitable, preferably, for administration as drops (oral drops), spray (oral spray) or syrup.

Said composition of the invention in water-based liquid form for the administration in the form of drops to paediatric subjects can be administered as such (by means of a dropper or by means of a spoon in which the drops are laid) or, alternatively, mixed with the milk to be administered to the paediatric subject. In the case where the composition of the invention in water-based liquid form is mixed with artificial milk, it also offers the advantage of reducing the foam that is formed in the preparation of milk through the step of mixing powdered milk and water.

Forming an object of the invention is a composition of the invention, comprising a simethicone and sucrose esters in the various embodiments of the invention, wherein said composition is for use as a medicament.

Forming an object of the invention is a composition of the invention, comprising simethicone and sucrose esters in the various embodiments of the invention, wherein said composition is for use in a method for the preventive and/or curative treatment of diseases of the gastrointestinal tract and disorders or symptoms related to these diseases, in subjects in need; preferably diseases, disorders or symptoms of the gastrointestinal tract associated with or related to a state of formation of gas in the gastrointestinal tract.

Preferably, said disorders of the gastrointestinal tract are intestinal colics or neonatal colics, abdominal cramps, flatulence, tympanites, aerophagia, swelling of the abdomen or gastrointestinal tract, dyspeptic disorders (or dyspepsia), disorders that require a reduction in the gastric emptying time.

Furthermore, the composition of the invention in the embodiment comprising simethicone, sucrose esters and a polysaccharide, such as for example xanthan gum, may be for use in a method for the treatment of gastroesophageal reflux.

In an embodiment, the composition of the invention is for use in a method for the treatment of intestinal or abdominal colics and/or abdominal cramps in paediatric subjects from 0 to 24 or 18 months, preferably in paediatric subjects from 0 to 12 months, more preferably from 0 to 6 months (for example 1 month, 2 months, 3 months, 4 months or 5 months), or neonates from 0 to 4 weeks with neonatal colics.

As a matter of fact, the combination of simethicone and sucrose esters enhances the effectiveness of the composition of the invention in breaking the foam generated by the proteins ingested by the subject, in particular by milk proteins (effect of increasing effectiveness or synergy). Thus, the composition of the invention is capable of treating the disorders caused by or related with the increase in the pressure in the gastrointestinal tract, in particular colics, intestinal or abdominal colics and/or abdominal cramps that occur in infants and/or children during the natural or artificial lactation period.

According to an embodiment, the composition of the invention comprises per “dose unit” (for example consisting of 10 drops to 30 drops, preferably 20 drops, of the composition according to Table 1, wherein 1 drop=about 0.5 ml): (a) simethicone as such at an amount comprised in the range from 10 mg to 100 mg, preferably from 20 mg to 60 mg, more preferably from 30 mg to 50 mg (for example 40 mg); (b) sucrose esters of fatty acids at an amount comprised in the range from 1 mg to 15 mg, preferably from 2 mg to 15 mg, more preferably from 5 mg to 15 mg (for example 10 mg).

Said “dose unit” of about 20 drops of the composition of the invention according to Table 1 corresponds to about 1 ml (0.5 ml/drop) of said composition in the form of a water-based liquid.

Said “dose unit” of the composition of the invention (for example from 10 drops/day to 30 drops/day, preferably 20 drops/day (or 1 ml), of the composition according to Table 1) can be administered to a subject in need once or twice a day, or divided into 2 to 4 portions to be administered during the 24 hours, depending on the type of dosage form and the needs of the subject.

Forming an object of the present invention is a method for the preventive and/or curative treatment of diseases of the gastrointestinal tract and of disorders or symptoms associated with these diseases, which provides for the administration of a therapeutically effective amount of the composition of the invention to a subject in need. Preferably, said treatment method treats diseases, disorders or symptoms of the gastrointestinal tract associated with or related to a condition of gas formation in the gastrointestinal tract, such as, for example, intestinal colics, preferably in neonates or children, abdominal cramps, flatulence, swelling of the gastrointestinal tract, dyspeptic disorders (or dyspepsia), disorders that require a reduction in gastric emptying time.

The term “therapeutically effective amount” refers to the amount of active compound and/or mixture of active compounds that elicits the biological or medicinal response in a tissue, system, mammal, or human being that is sought and defined by an individual, researcher, veterinarian, physician, or other clinician or health worker.

In the context of the present invention, the expression “subjects” is used to indicate human subjects or animal subjects (e.g. pets, such as dogs or cats or other mammals). Preferably, the compositions of the invention are for use in treatment methods for human subjects.

In the context of the present invention, the term “neonate” is used to indicate a mammal (human or animal subject) in the period comprised from the time of birth to the first 4 weeks of life.

In the context of the present invention, the term “paediatric subject” is used to indicate a mammal (human or animal subject) preferably in the period from 1 month to 18 months, more preferably from 1 month to 12 or 6 months.

Lastly, forming an object of the present invention is the use (non-therapeutic use) of the composition of the invention comprising a mixture comprising, or alternatively, consisting of a simethicone and sucrose esters according to the various embodiments of the invention as an antifoam agent. When the use of the composition of the invention as antifoam is for purposes other than pharmaceutical or food purposes, for example for non-therapeutic purposes in general, it is clear that the composition of the invention may comprise additives and/or excipients other than those of pharmaceutical or food grade.

In a preferred embodiment of non-therapeutic use of the composition of the present invention, said composition (comprising a simethicone and sucrose esters according to any one of the described embodiments) is for use as an antifoam in the preparation of a liquid food (for example artificial milk) comprising water and a composition comprising milk proteins (e. g. powdered milk), preferably for paediatric subjects (preferably 0-12 months and/or 12-18 months) or neonates. Advantageously, the addition of the composition of the invention in water-based liquid form to a mixture of water and powdered milk prevents the formation of foam which generally forms at the liquid-air interface in the preparation of the artificial milk (foam caused by milk proteins).

An example of a method for the preparation of a liquid food based on milk proteins, for example artificial milk, preferably for paediatric subjects, in the presence of the composition according to the invention comprises the following steps:

(i) heat a suitable amount of water (for example from 20 ml to 50 ml) at a temperature of 40° C.-50° C. to obtain a liquid of step (i);

(ii) add to said liquid of step (i) a composition according to the invention (for example from 1 ml to 2 ml of the composition of Table 1) to obtain a liquid of step (ii);

(iii) add to said liquid of step (ii) a suitable amount of a product comprising milk proteins, for example powdered milk (such as from 2 g to 10 g), and stir until the powder is completely dissolved.

Alternatively, said method for the preparation of a liquid food based on milk proteins, for example artificial milk, may provide firstly adding a product comprising milk proteins (for example powdered milk) to water and then the composition of the invention to said mixture of water and a product comprising milk proteins.

Furthermore, the present invention relates to a kit for the preparation of a liquid food (for example artificial milk), wherein said kit comprises a product comprising milk proteins (for example powdered milk) and a composition of the invention (according to any one of the embodiments and aspects described), preferably wherein said liquid food is for paediatric subjects (preferably 0-12 months and/or 12-18 months) or neonates (0-4 weeks).

Lastly, forming an object of the present invention is a liquid food (for example artificial milk), wherein said food comprises water, a product comprising milk proteins (for example powdered milk) and the composition of the invention (according to any one of the embodiments and aspects described), preferably wherein said liquid food is for paediatric subjects (preferably 0-12 months and/or 12-18 months) or neonates (0-4 weeks).

The composition of the invention, comprising a simethicone and sucrose esters in the various embodiments of the invention may be a pharmaceutical composition, medical device composition, nutraceutical composition, dietary supplement product or food for special medical purposes (in short, FSMP)

In the context of the present invention, the expression “medical device” is used in the meaning according to the Italian Legislative Decree n° 46 dated 24 Feb. 1997 or according to the new Medical Device Regulation (EU) 2017/745 (MDR).

For the sake of clarity, with the aim of achieving the object of the present invention, the components (or active components) of the mixture of the present invention (a) simethicone and (b) sucrose esters may also be administered separately (preferably at a time interval ranging between 30 minutes and 60 minutes) and in any order but, preferably, (a) and (b) are administered to a subject simultaneously, even more preferably in a single composition so as to obtain a more rapid effect and for ease of administration. When the active ingredients (a) and (b) are administered in a single composition, said single composition corresponds to the composition of the present invention.

The composition of the invention optionally comprises said at least one food or pharmaceutical grade additive and/or excipient, i.e. a substance devoid of therapeutic activity suitable for pharmaceutical or food use. In the context of the present invention the additives and/or excipients acceptable for pharmaceutical or food use comprise all auxiliary substances known to the man skilled in the art for the preparation of compositions in solid, semi-solid or liquid form such as for example diluents, solvents (including water, glycerine, ethyl alcohol), solubilisers, acidifiers, thickeners, sweeteners, flavour enhancers, dyes, lubricants, preservatives, pH stabilising buffers and mixtures thereof. Preferred examples of such substances are sodium benzoate, sucralose, xanthan gum, citric acid, potassium sorbate and/or natural or artificial flavours.

Unless specified otherwise, the expression composition or mixture comprising a component at an amount “comprised in a range from x to y” is used to indicate that said component can be present in the composition or mixture at all the amounts present in said range, even though not specified, extremes of the range comprised.

Table 1 reports an embodiment of an aqueous liquid composition in the form of a suspension for oral administration as drops according to the present invention (% by weight with respect to the total weight of the composition), reported solely for illustrative and thus non-limiting purposes.

In Table 1 the amounts (mg/drop and %) refer to amounts per drop. The average daily intake for a child from 0 to 12 or 18 months is advantageously from 10 to 30 drops a day, preferably 20 drops once or twice a day.

TABLE 1

| Compound name | mg/drop | % |
|---|---|---|
| Simethicone emulsion 30% w/w | 6.67 (2) | 13.33 (4) |
| *(simethicone as such) | | |
| Sodium benzoate | 0.0500 | 0.10 |
| Sucrose esters** (or sucrester) | 0.5000 | 1.00 |
| Additives and/or excipients | 0.2100 | 0.42 |
| Demineralised water | 42.57 | 85.15 |
| | 50.00 | 100 |

*30% by weight of simethicone with respect to the weight of the simethicone-water emulsion.
**sucrose esters for example SP70 ‘473’ (Brenntag) having for example the following composition by weight: sucrose esters of fatty acids of at least 90% (70% stearic acid or palmitic acid monoesters); free fatty acids not exceeding 3%; free sucrose not exceeding 2%; maximum p-methoxyphenol 100 micrograms/kg; maximum acetaldehyde 50 ppm; humidity not exceeding 4%; acid value not exceeding 5 expressed as mq of KOH/g.

Further embodiments (FR-i, FR-ii, FR-iii, FR-iv) of the composition according to the invention are reported in Table 2.

TABLE 2

| | FR-i | FR-ii | FR-iii | FR-iv |
|---|---|---|---|---|
| Compound name | % w/w | % w/w | % w/w | % w/w |
| Simethicone emulsion | 15.0 (4.5) | 20.0 (6) | 15.0 (4.5) | 12.0 (3.6) |
| 30% w/w * (simethicone | | | | |
| as such) | | | | |
| Sucrose esters** (or | 1.5 | 1.5 | 1.0 | 1.0 |
| sucrester) | | | | |
| Xanthan gum | 0.5 | — | — | 0.5 |
| Karaya gum | — | 0.5 | — | — |
| Tara gum | — | — | 0.5 | — |
| Additives and/or excipients | 0.4 | 0.5 | 0.8 | 1.0 |
| Demineralised water | 82.6 | 77.5 | 82.7 | 85.5 |
| | 100 | 100 | 100 | 100 |

* and **as per Table 1.

Embodiments (FRn) of the present invention are outlined below:

FR1. A composition comprising, or alternatively, consisting of simethicone and sucrose esters (or sucrester) and, optionally, said composition comprises at least one food or pharmaceutical grade additive and/or excipient.

FR2. The composition according to FR1, wherein said composition is in liquid form; preferably in aqueous solvent.

FR3. The composition according FR 1 or 2, wherein said composition is for oral use; preferably for administration as drops, spray or syrup.

FR4. The composition according to any one of the FRs 1 to 3, wherein simethicone and sucrose esters are in a weight ratio, expressed as simethicone as such toward sucrose esters comprised in the range from 15:1 to 1:1; preferably 5:1 to 3:1, more preferably 4:1.

FR5. The composition according to any one of FRs 1 to 4, wherein said composition comprises, with respect to the total weight of the composition, simethicone as such at a by weight % comprised in the range from 0.5% to 10%, preferably from 1% to 7%, more preferably from 3% to 5%, and esters of fatty acids at a by weight % comprised in the range from 0.1% to 5%, preferably from 0.5% to 2%, more preferably from 0.8% to 1.2%.

FR6. The composition according to any one of FRs 1 to 5, wherein said composition is for use as a medicament.

FR7. The composition according to anyone of FRs 1 to 5, wherein said composition is for use in a method for the treatment of the gastrointestinal tract and disorders or symptoms associated thereto, in needy subjects; preferably diseases, disorders or symptoms of the gastrointestinal tract associated or relating to a state of formation of gas in the gastrointestinal tract.

FR8. The composition according to FR7, wherein said composition is for use in a method for the treatment of intestinal colics, abdominal cramps, flatulence, tympanites, aerophagia, swelling of the abdomen, dyspeptic disorders (or dyspepsia), disorders that require the reduction of the gastric emptying time.

FR9. The composition according to FR 8, wherein said composition is for use in a method for treatment of intestinal colics and/or abdominal cramps in paediatric subjects between 0 and 24 months, preferably in paediatric subjects between 0 and 12 months, more preferably between 0 and 6 months.

FR10. Use of the composition according to any one of FRs 1 to 5 for non-therapeutic purposes as anti-foam agent.

### Experimental Part I

Purpose: evaluation of an antifoam effect of a composition according to the invention

### Materials and Methods

Compositions Under Analysis

Composition 1: composition with antifoam according to Table 1 (composition according to the invention, comprising simethicone and sucrose esters of fatty acids);

Composition 2: composition without antifoam, i.e. composition not comprising simethicone and sucrose esters of fatty acids (comparative composition).

Method

In order to determine the antifoam activity of the compositions under analysis, the Minijet™ device (an instrument manufactured by Teclis) was used applying the following method:

1. Creation of liquid jet foam up to a predefined initial foam value (160 cm³ in this experiment) (the liquid jet runs throughout the entire experiment at the same flow rate: 900 cm³/min); zone 1 in FIG. 1 (FIG. 1, representation of a standard antifoam composition).

2. Injection of the composition under analysis (for example, Composition 1 or 2, amount 6 ml) into the jet itself; point A in FIG. 1.

3. Foam knock down; knock down is directly linked to the efficiency of the composition under analysis in breaking the foam.

4. In order to know the duration (persistence) of the composition under analysis in knocking down the foam (antifoam effect), the time required to return to the initial foam value (volume of the foam when the composition under analysis was injected (point A, i.e. 160 cm³)) is analysed. The time between point A and point B (zone 2 of FIG. 1) is called the “durability of the antifoamer”.

After 3000 seconds of experiments, the initial foam value is not restored.

The samples were prepared at 50° C. and the temperature of the experiment was 50° C. (pressure 1 atm).

### Results

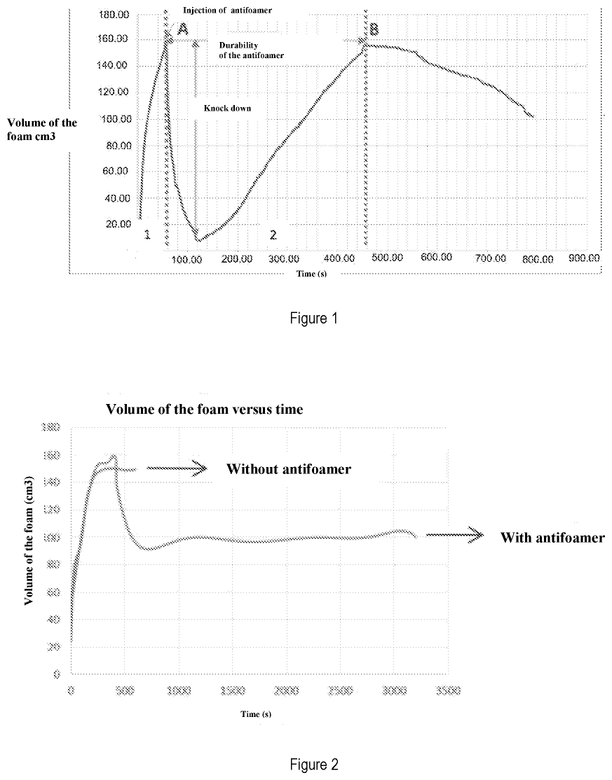

The volumes of foam with respect to time measured for Composition 1 and Composition 2 are shown in FIGS. 2, 3 and 4.

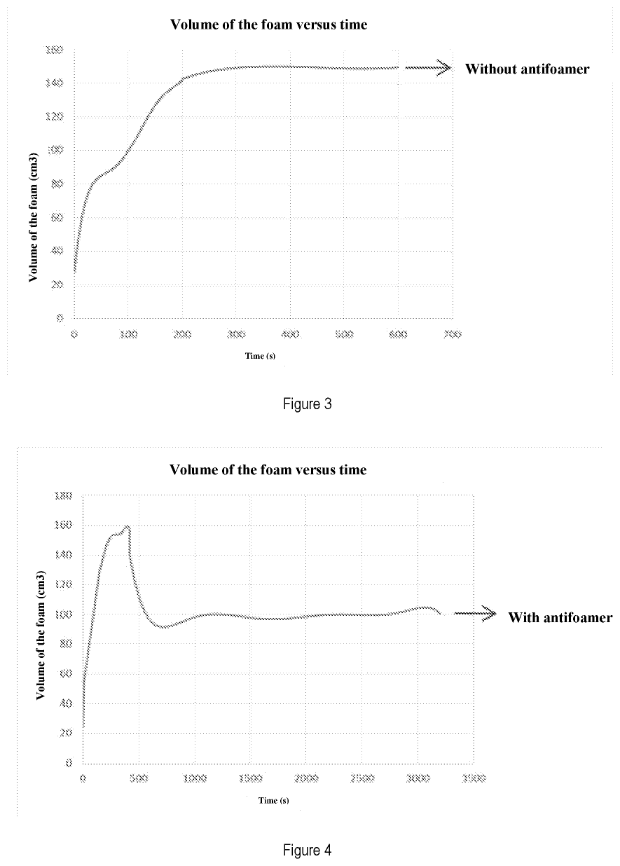

As reported in FIGS. 2 and 4 (comparison between Composition 1 and 2), Composition 1 (composition according to the invention) knocked down 68 cm³ of foam within a period of time of about 2800 seconds, while the Composition 2 (comparative composition) did not knock down the foam (FIGS. 2 and 3).

### Experimental Part II

1. Purpose: evaluation of the reduction in the volume of foam formed on the surface of a water and powdered milk mixture using a composition according to the invention (comprising a simethicone and a sucrester) as compared to compositions comprising simethicone alone (in a water-based liquid or in an oil-based liquid).

### 2. Material

Water and powdered milk mixture: prepared by dissolving 4.33 g of powdered milk in 30 ml of water;

The “powdered milk” used (Crescendo baby 2, powdered milk for continuation from more than 6 to 12 months, Coop) has for example the following composition: Demineralised milk serum, Skimmed milk, Vegetable oils (sunflower seed oil, rapeseed oil and extra virgin olive oil), Milk cream, Maltodextrins, Mineral salts (calcium phosphate, potassium citrate, calcium carbonate, sodium chloride, calcium citrate, potassium chloride, magnesium citrate, iron lactate, zinc sulfate, copper sulfate, potassium iodide, manganese sulfate, sodium selenite), Vitamins (C, pantothenic acid, E (milk), niacin, A, B6, B1, folic acid, K1, biotin, D3), Choline bitartrate, Emulsifier: soy lecithin, Inositol, Taurine, L-carnitine, Antioxidant: extract rich in tocopherol. Quantitative composition for 100 g of powdered milk: Fats 26.4 g (including saturated fatty acids 9.6 g and polyunsaturated fatty acids 5.4 g), carbohydrates 54.7 g, proteins 11.7 g, fibres 0 g, remaining vitamins and mineral salts.
Composition 1 (PM47-2; comparative): simethicone in water-based solution (concentration of simethicone (as such): 40 mg/ml).
Composition 2 (comparative): simethicone in olive oil-based solution (concentration of simethicone (as such): 40 mg/ml; olive oil composition: standard)
Composition 3 (PQ85-1; according to the present invention): comprising a simethicone and a sucrester according to Table 1 (equivalent to a simethicone concentration (as such): 40 mg/ml)

### 3. Method

The Water and powdered milk mixture was prepared by carrying out the following steps: heat 30 ml of water to a temperature of 40° C.-50° C. (pressure 1 atm), add 4.33 g of powdered milk and stir until the powder is completely dissolved.

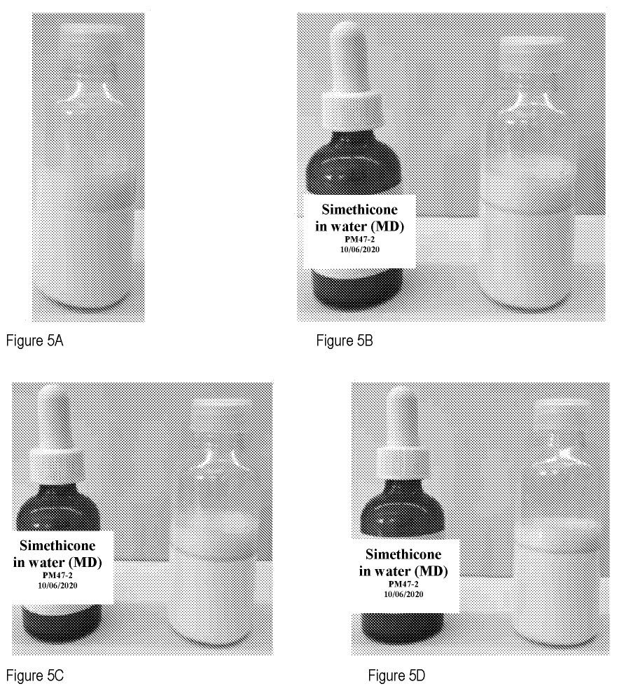

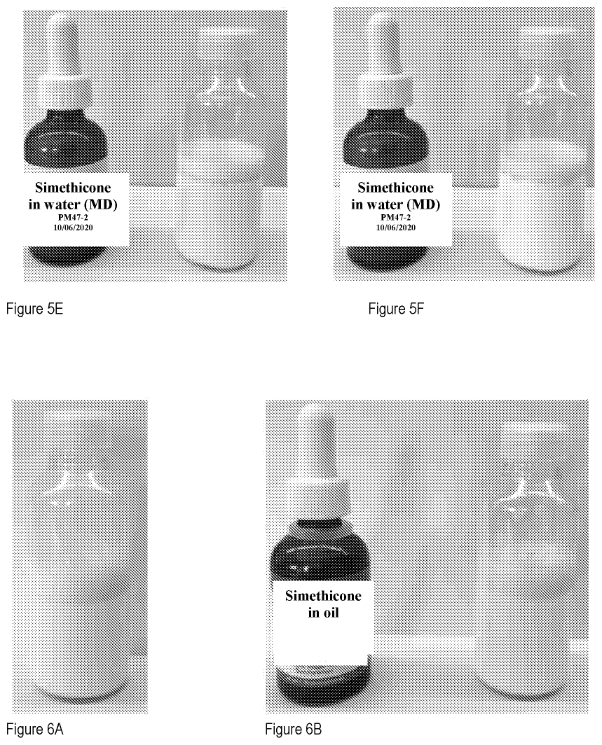

Said Water and powdered milk mixture has a foam at the liquid-air interface (FIGS. 5A, 6A, 7A). One of the compositions under analysis (Compositions 1-3) was added to said Water and powdered milk mixture. The Compositions 1, 2 and 3 were added to said Water and powdered milk mixture in an amount such as to dose the same quantity of simethicone in the 3 tests: 40 mg/ml.

The state of the Water and powdered milk mixture was photographed every 2 minutes (t₀, t1, t2, t3, t4) from the zero time of addition of the composition under analysis to the time of 8 minutes, in order to visually verify the foam reduction at the liquid-air interface and measure said reduction as a volume/volume percentage for example using a graduated cylinder (volume occupied by the foam).

### 4. Results

### Visual Evaluation of Foam Reduction:

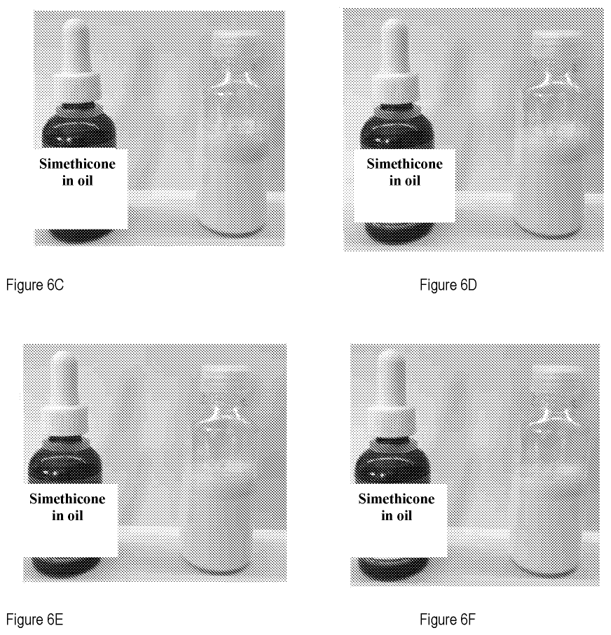

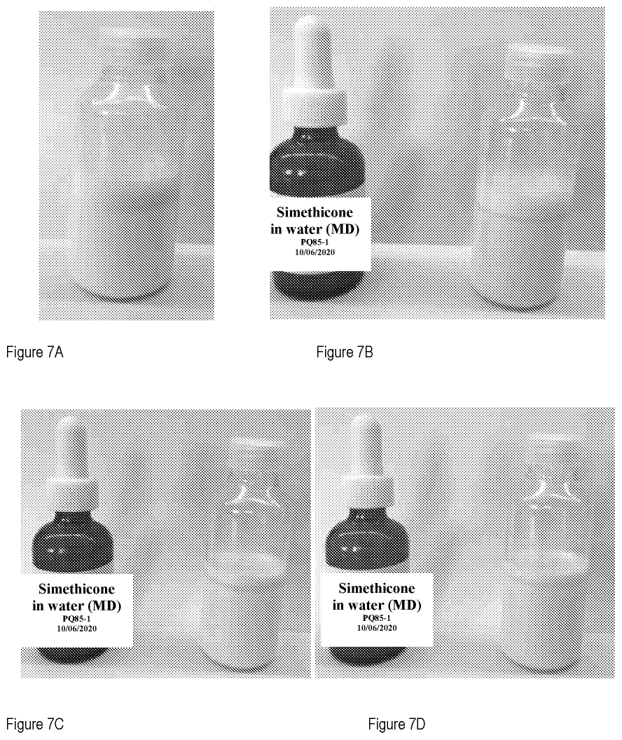

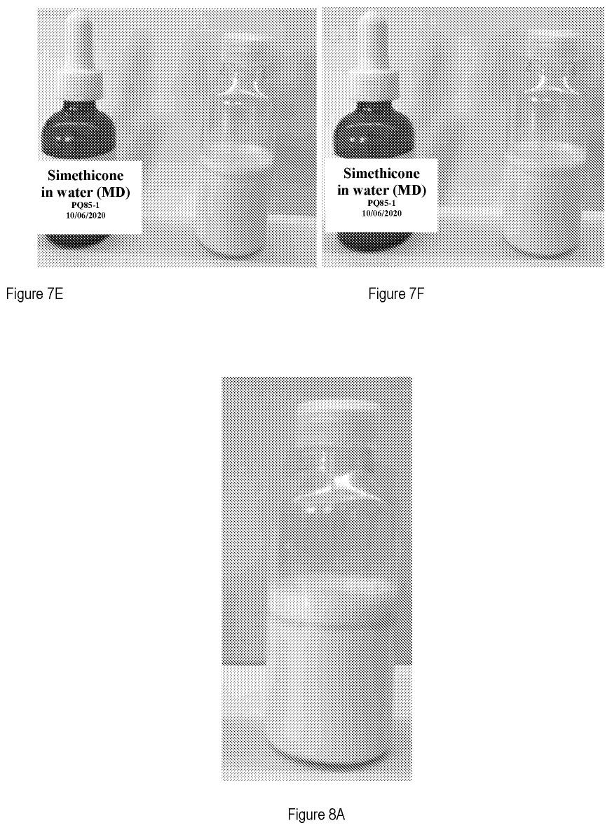

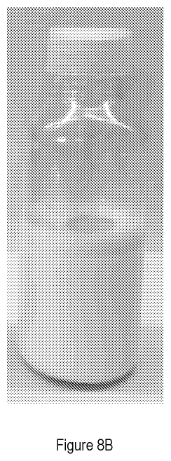

Composition 1 (simethicone in water; PM47-2): FIGS. 5A, 5B, 5C, 5D, 5E, 5F;
Composition 2 (simethicone in oil): FIGS. 6A, 6B, 6C, 6D, 6E, 6F;
Composition 3 (simethicone+sucrester; PQ85-1): FIGS. 7A, 7B, 7C, 7D, 7E, 7F.

As observable from the aforementioned FIGS. 5, 6 and 7, the composition according to the invention (Composition 3, simethicone+sucrester; PQ85-1) shows a greater effectiveness in reducing the foam at the liquid-air interface than compositions 1 and 2 comprising only simethicone, demonstrating the greater/synergistic effect of the association of the sucrester with simethicone.

Furthermore, from FIGS. 8A and 8B respectively, it is observable that Composition 1 (simethicone in water) is positioned at the interface between the liquid phase and the foam, while Composition 2 (simethicone in oils) is positioned over the foam; this partly explains the reduced capacity of these compositions to reduce the volume of the foam.

% volume/volume reduction of foam
Composition 1 (simethicone in water; PM47-2): about 43%;
Composition 2 (simethicone in oil): about 25%;
Composition 3 (simethicone+sucrester; PQ85-1): about 75%.

### Experimental Part III

1. Purpose: to evaluate the ability to inhibit the formation foam during reconstitution of the powdered milk (such as milk to be administered to a paediatric subject) in the presence of a composition according to the invention (comprising a simethicone and a sucrester) or in the absence of said composition according to the invention.

2. Material
Powdered milk (Crescendo baby 2, powdered milk for continuation from more than 6 to 12 months, Coop) having for example the following composition: Demineralised milk serum, Skimmed milk, Vegetable oils (sunflower seed oil, rapeseed oil and extra virgin olive oil), Milk cream, Maltodextrins, Mineral salts (calcium phosphate, potassium citrate, calcium carbonate, sodium chloride, calcium citrate, potassium chloride, magnesium citrate, iron lactate, zinc sulfate, copper sulfate, potassium iodide, manganese sulfate, sodium selenite), Vitamins (C, pantothenic acid, E (milk), niacin, A, B6, B1, folic acid, K1, biotin, D3), Choline bitartrate, Emulsifier: soy lecithin, Inositol, Taurine, L-carnitine, Antioxidant: extract rich in tocopherol. Quantitative composition for 100 g of powdered milk: Fats 26.4 g (including saturated fatty acids 9.6 g and polyunsaturated fatty acids 5.4 g), carbohydrates 54.7 g, proteins 11.7 g, fibres 0 g, remaining vitamins and mineral salts.
Composition 3 (PQ85-1; according to the present invention): comprising a simethicone and a sucrester according to Table 1.

3. Method

3.1. Reconstitution of powdered milk in the presence of the composition according to the invention Steps:

(i) Heat 30 ml of water to a temperature of 40° C.-50° C. (pressure of 1 atm) to obtain a liquid of step (i);

(ii) add to said liquid of step (i) 1 ml of Composition 3 to obtain a liquid of step (ii);

(iii) add 4.33 g of powdered milk to said liquid of step (ii) and stir until the powder is completely dissolved.

3.2. Reconstitution of powdered milk in the absence of the composition according to the invention Steps:

(i) heat 30 ml of water to a temperature of 40° C.-50° C. to obtain a liquid of step (i); (iii) add 4.33 g of powdered milk to said liquid of step (i) and stir until the powder is completely dissolved.

4. Results

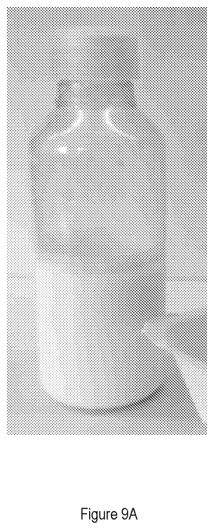

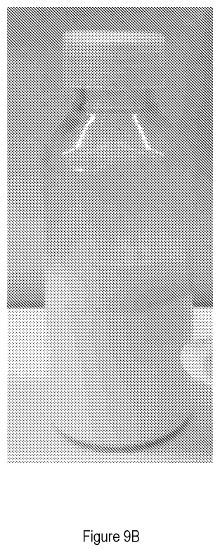

During the reconstitution of the powdered milk in the presence of the composition according to the invention (FIG. 9A), the formation of a reduced amount of foam (foam formation prevention action) is observed with respect to the similar reconstitution of the powdered milk in the absence of the composition according to the invention (FIG. 9B).

### Experimental Part IV_ Defoaming Test

1. Purpose: to evaluate the ability of a composition according to the invention (comprising a simethicone and a sucrester) to reduce the foam of a foaming solution compared to a composition comprising simethicone alone.

2. Material

2.1. Foaming solution: 2.5 g of sodium docusate (bis(2-ethylhexyl) sodium sulfosuccinate) were dissolved in 500 ml of water (heat to 50° C. if necessary, at a pressure of 1 atm).

2.2. Tested compositions:
Comparison composition: simethicone in water (simethicone as such: 66.6 mg/ml, other ingredients: citric acid monohydrate; sodium citrate; methyl hydroxypropyl cellulose; carboxypolymethylene; saccharin; sodium benzoate; sorbic acid; sodium bicarbonate; essences; purified water).
Composition 3 (PQ85-1; according to the present invention): comprising a simethicone and a sucrester according to Table 1 (simethicone (as such): 40 mg/ml).

2.3 Machine: Flash shaker SF1 at 500 osc/min.

3. Method (the method used is an adaptation of the one reported in European Pharmacopoeia in the Simethicone monograph [07/2011:1470]): 100 ml of foaming solution (point 2.1) were placed in a 250 ml cylinder and one of the tested compositions (Composition 1 or 3) was added. Stirring was applied using “Flash shaker SF1” for 10 seconds, and then the time elapsed up to the occurrence of the first liquid phase without foam appeared (in short, Foam reduction time) was measured.

4. Results

Foam reduction times
Comparison composition: simethicone in water): 4 seconds
Composition 3 (simethicone+sucrester, according to the invention): 1 second

1. A method for the treatment of diseases or symptoms associated with or related to a condition of gas formation in the gastrointestinal tract in a subject in need thereof, comprising administering to the subject a composition comprising a mixture comprising or, alternatively, consisting of simethicone and sucrose esters of fatty acids (or sucrester) and, optionally, said composition comprises at least one food or pharmaceutical grade additive and/or excipient.

2. The method according to claim 1, wherein said composition further comprises a polysaccharide, wherein said polysaccharide is a gum having an average molecular weight comprised from 500 kDa to 10000 kDa, preferably from 700 kDa to 5000 kDa, more preferably from 1000 kDa to 2500 kDa.

3. The method according to claim 2, wherein said polysaccharide is selected from a group comprising or, alternatively, consisting of: xanthan gum, gum karaya, gum arabic, tara gum and mixtures thereof; preferably said polysaccharide comprises or, alternatively, consists of xanthan gum.

4. The method according to claim 1, wherein said subject has intestinal colics, abdominal cramps, flatulence, tympanites, aerophagia, swelling of the abdomen, dyspeptic disorders or dyspepsia, disorders that require the reduction of the gastric emptying time.

5. The method according to claim 1, wherein said composition is for use in a method for treatment of intestinal colics and/or abdominal cramps in paediatric subjects or neonates, preferably in paediatric subjects from 0 to 12 months or 18 months, more preferably from 0 to 6 months or from 0 to 4 weeks.

6. The method according to claim 1, wherein the composition is administered orally.

7. The method according to claim 1, wherein said composition is in liquid form.

8. The method of claim 7, wherein said composition in liquid form is a water-based composition.

9. The method according to claim 6, wherein said composition is formulated as oral drops, oral spray or syrup.

10. The method according to claim 8, wherein said composition in water-based liquid form is administered to a paediatric subject in a mixture with an artificial milk, wherein said artificial milk comprises water and powdered milk.

11. The method according to claim 1, wherein said composition comprises in a percentage by weight with respect to the total weight of the composition, simethicone as such from 1% to 10% and fatty acid esters from 0.05% to 1.5%; preferably simethicone as such from 1% to 7% and fatty acid esters from 0.5% to 1.5%, more preferably simethicone as such from 3% to 5% and sucrose esters of fatty acids 0.8% to 1.2%.

12. The method according to claim 1, wherein said simethicone and said sucrose esters of fatty acids are in a weight ratio, expressed as [simethicone as such: sucrose esters of fatty acids] comprised in the range from 15:1 and 1:1; preferably from 5:1 to 3:1, more preferably 4:1.

13. The method according to claim 1, wherein said sucrose esters of fatty acids comprise fatty acid sucrose monoesters having a C16 and/or C18 number of carbons in a percentage by weight from 50% to 90%, preferably from 60% to 80%.

14. Use for non-therapeutic purposes of a composition according to claim 1 as an antifoam agent for the preparation of a liquid food comprising water and a product comprising milk proteins.

15. A kit for preparing a liquid food comprising or, alternatively, consisting of:
a product comprising milk proteins, preferably powdered milk, more preferably powdered milk for paediatric subjects or neonates, and
a composition according to claim 1.

16. A composition comprising a mixture comprising or, alternatively, consisting of a simethicone and sucrose esters of fatty acids (or sucrester),
wherein said composition comprises, in a percentage by weight with respect to the total weight of the composition, simethicone as such from 1% to 10% and fatty acid esters from 0.05% to 1.5%,
and, optionally, said composition comprises at least one food or pharmaceutical grade additive and/or excipient.

17. The composition according to claim 16, wherein said composition comprises in a percentage by weight with respect to the total weight of the composition, simethicone as such from 1% to 7% and fatty acid esters from 0.5% to 1.5%, more preferably simethicone as such from 3% to 5% and sucrose esters of fatty acids 0.8% to 1.2%.

18. The composition according to claim 16, wherein said composition further comprises a polysaccharide, wherein said polysaccharide is a gum having an average molecular weight comprised from 500 kDa to 10000 kDa, preferably from 700 kDa to 5000 kDa, more preferably from 1000 kDa to 2500 kDa; preferably wherein said polysaccharide is selected from a group comprising or, alternatively, consisting of: xanthan gum, gum karaya, gum arabic, tara gum and mixtures thereof.
